(19) United States
(12) Patent Application Publication
(10) Pub. No.: US 2004/0192996 A1
(43) Pub. Date: Sep. 30, 2004

(54) Device for recording dream recollections

(76) Inventor: Tracy Catherine Lemmon, San Francisco, CA (US)

Correspondence Address: Bernard Berman, Esq., Suite 700, 1220 W 6th Street, Cleveland, OH, 44113, US

(21) Appl. No.: 10/810,953

(22) Filed: Mar. 26, 2004

Related U.S. Application Data
(60) Provisional application No. 60/457,837, filed on Mar. 26, 2003.

(51) Int. Cl. A61N 5/00

(52) U.S. Cl. 600/1; 128/897

(57) ABSTRACT

A device for recording dream recollections is provided. Embodiments of the device encompass an audio recording and storage means for recording, storing, replaying and otherwise manipulating one or more audio files. Such embodiments provide a person the ability to conveniently record dream recollections by speaking such recollections aloud after activating the device. Embodiments of the device can sit on a table proximate a bed, or be attached to a wall, bedpost, or headboard in a removable or fixed manner. Activation of such embodiments of the device can be via an activation means that is configured to be readily located in the dark or low ambient light conditions. Some embodiments of the present invention are coupled to other devices such as a timing device such as clock or clock radio, where such timing device can provide automatic activation.

### CROSS-REFERENCE TO RELATED APPLICATIONS

This application claims priority to U.S. Provisional Application No. 60/457,837, entitled “Dream Recording Device,” filed Mar. 26, 2003.

### TECHNICAL FIELD

The present invention relates generally to a device for recording dream recollections, and more specifically to such a device that can electronically record a verbal record of a person's dream upon waking.

### BACKGROUND

People have always been interested in the interpretation of their dreams. To this effect, many books have been written to assist such people in a process of self-interpretation. In addition, people often consult therapists or doctors about their dreams to seek help in gaining some insight into their subconscious minds through the interpretation of such dreams. It is not unusual for people to use their dreams as a source of inspiration in their daily lives, for invention, story ideas, and the like.

However, the interpretation of dreams is often hindered by a person's inability to remember a dream for more than a few moments after waking. This problem has been recognized for quite some time, but without a readily implemented solution.

For example, some prior art devices are configured to detect a person's REM (Rapid Eye Movement) dream state and when such state is detected, provide to the person some form of feedback. The feedback serves to alert the person that a dream is occurring to hopefully result in a lucid dream that may be more memorable than had the person not been alerted. Such a device is disclosed in U.S. Pat. No. 5,507,716 to LaBerge et al., entitled “EQUIPMENT AND METHODS USED TO INDUCE LUCID DREAMS IN SLEEPING PERSONS.” However, the '716 patent teaches the use of equipment that is attached to, or worn by the person (see, Abstract) to detect the REM dream state and the process of alerting the person may result in the person waking, thus interfering with the person's sleep, or even alter the nature of any dream the person experiences. In addition, the '716 patent does not disclose or suggest recording the details of any dreams, lucid or not. Rather it is only directed to helping persons have lucid dreams as LaBerge et al. suggest that lucid dreaming may be beneficial to a person's good health and other qualities of his or her life (see, col. 5, line 60 through col. 6, line 18). Hence such a device seems directed to infrequent use, for example as a device directed to medical usages for the study of sleep related problems.

More recently, a device that does not rely on sensing a dream state was disclosed in U.S. Pat. No. 6,540,664, entitled “APPARATUS FOR FACILITATING ANALYSIS OF DREAM ACTIVITY” to Blair. Rather than sensing REM sleep, the device of the '664 patent employs at least two micro controllers to calculate the expected occurrence of a person's REM sleep events (see, Abstract) and to control the functions of the device. In addition, the device is also programmed to wake the user during such calculated REM sleep events by flashing LEDs, or other means, in a seemingly similar manner to that of the '716 patent. Once the person is awakened, a dictation system is provided to allow the person to orally record the details of the dreams that were happening when he or she was awakened. While being wakened during a dream may provide for excellent recall of a dream's details, a person generally experiences several REM periods during a night's sleep and thus is likely to be woken by the '664 device during each of those periods. Thus while using the '664 device, a normal, complete night's sleep may be problematic. In addition, since the '664 device is shown as being fixably mounted so that is positioned above a person while that person is asleep, it can be distracting and actually interfere with such sleep (see, FIG. 1). Further, even though the '664 device provides a voice activated dictation means, the complexity of programming the device to calculate REM periods seem likely to make such a device difficult to use. Hence it is believed that the '664 device, like the device of the '716 patent, seems directed to infrequent use, for example as a device directed to medical usages for the study of sleep related problems.

Thus, individuals interested in investigating their dream recollections on a regular basis without being attached to any devices that will enhance the REM state and perhaps provide extraordinary dream recollections, or without being intentionally woken up either during or shortly after a REM event and thus have only an interrupted sleep periods and who require an easy to use, and relatively inexpensive device, have been limited to traditional recording means not specifically directed to recording dream recollections.

For example, a person can keep paper and a writing instrument on a bedside table for recording dream recollections. However, this can be inconvenient, especially where a person wakes after a dream in the middle of the night and has to turn on a light to use the paper and writing instrument. Even if this not the case and sufficient light to find the paper and instrument is available, the person might have to find eye glasses or insert contact lenses to effectively write out the recollection of the dream on a piece of paper. In addition, the activity described above that may be needed to locate the materials and prepare oneself to write out the dream recollection on paper can take a person's mind away from the dream and result in forgetting portions of the dream before it is accurately transcribed onto the paper.

Another known recording device useable for recording dream recollections is a conventional tape recorder. Like the paper and writing instrument discussed above, the tape recorder can be kept on a bedside table, but also like the paper and writing instrument the conventional tape recorder can be inconvenient and/or difficult to use when just awakening. For example, such devices are generally made with a requirement to depress at least two buttons to activate the recording mode. Thus, if the person wakes in the middle of the night, he or she will likely have to turn on a light not only to find the recorder but also to find the correct buttons to turn on the record function. Also it is well known that a person's motor skills are often diminished immediately upon wakening thus for many people it may be difficult to turn on the device's record mode even if the multiple buttons are readily located.

A more recently available conventional option is the use of a standard digital voice recorder. Such devices are generally small, credit-card shaped and sized so that a person can easily fit it in his or her pocket. Such digital voice recorders are optimized for use by a person to record short notes or messages during the person's day for later replay. While digital voice recorders can have advantages over a conventional tape recorder, for example they do not normally require more than one button or switch to enter the record mode, using such a typical digital voice recorder to record one's dreams can be inconvenient. For example, its small size is likely to make it more difficult to locate on a bedside table in the dark than a conventional tape recorder or even the paper and writing instrument. And, while only a single button or switch may be required to enter the record mode, such controls are generally much smaller than those of conventional recorders as they are optimized for use during a person's day. Thus the standard digital recording device can be even more difficult than the conventional tape recorder to activate for recording dream recollections.

Therefore, it should be realized that it would be desirable to have a recording device suitable for recording dream recollections that does not suffer from the disadvantages of the devices and methods currently known.

### BRIEF DESCRIPTION OF THE DRAWINGS

Embodiments in accordance with the present invention are described below with reference to the following accompanying drawings.

FIG. 1 is a view of a device in accordance with embodiments of the present invention as seen from directly above the device; and

FIG. 2 is a view of the device of FIG. 1 as seen from a side corresponding to a long axis of the device.

### DETAILED DESCRIPTION

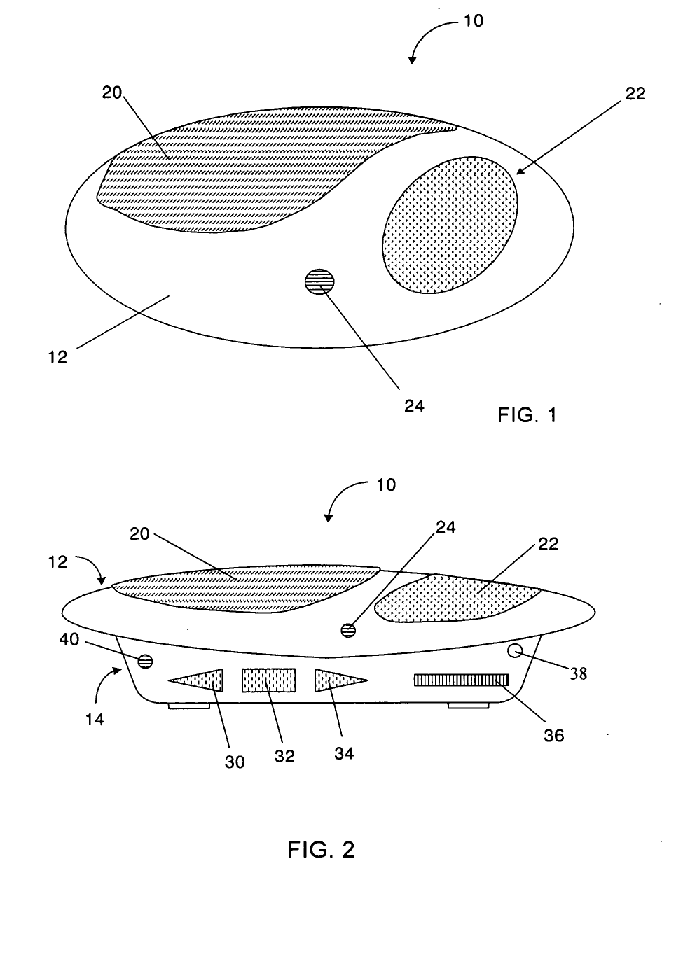

Exemplary embodiments in accordance with the present invention will be described with reference to the aforementioned figures. Various modifications, adaptations or variations of such exemplary embodiments, described herein, may become apparent to those skilled in the art as such embodiments are disclosed. It will be understood that all such modifications, adaptations or variations that rely upon the teachings of the present invention, and through which these teachings have advanced the art, are considered to be within the scope and spirit of the present invention. For example, while the embodiment depicted in FIGS. 1 and 2 has a generally oval shape and a particular placement of the various controls depicted, other embodiments of the present invention can have other shapes and other control placement while still being within the scope and spirit of the present invention.

Embodiments of the present invention include a housing unit constructed of any appropriate material currently known to one of ordinary skill in the art, or any appropriate material that will become known to one of ordinary skill in the art, provided that such material is appropriate for housing a small electronic device and functional controls of such electronic device. Furthermore, for some housing units, the aforementioned functional controls, i.e. a record activation button or switch, may be considered as being a portion of the housing unit, while for other housing units, such controls may be considered as a portion of the small electronic device contained therein. Such materials include, but are not limited to, plastic materials, metal materials, ceramic or glassy materials or combinations thereof.

Furthermore, while some embodiments of the present invention are of a size and shape such that it may sit on a bedside table or other piece of furniture in a stable fashion, or be portable, other embodiments can be made that are more suitable for being attached, fixably or removably, to another device or structure. For example, some embodiments of the present invention can be attached to a bed or a wall, or incorporated into or coupled to another device such as a radio, stereo, clock, clock radio, lighting fixture or the like.

Finally, depending upon the size and shape of an embodiment in accordance with the present invention, the housing unit of such embodiment can include a top surface, a bottom surface, a front portion, a back portion and/or one or more side portions.

Turning first to FIG. 1, a view of an embodiment of the present invention is provided where a dream recollection recording device 10 is seen as viewed from directly above such device. As depicted, device 10 encompasses an upper surface 12 which in turn encompasses an activation means 20, a sound emitting region 22 and a sound accepting region 24. It should be noted that while recording device 10 is depicted as having a generally oval profile, other shapes and/or configurations of device 10, while not shown, are possible and such other shapes and/or configurations will represent other embodiments in accordance with the present invention. It should be realized that while not shown, housing unit 14 contains therein an electronic device that encompasses an audio recording means, a recording storage means and a recording/storage control means.

Activation means 20 typically serves to both activate and deactivate the recording function of device 10. In some embodiments activation means 20 is an illuminated activation means where such illumination is provided by any appropriate method. For example, in some embodiments, activation means 20 is a depressible button or an illuminated switch that is formed in part or in its entirety of a photo luminescent material. Alternatively, illumination of means 20 is provided by one or more sources of illumination contained proximate to, or within means 20. Regardless of how activation means 20 is illuminated, such illumination can be of any color, but is most advantageously a soothing blue, green, violet, yellow, or red color and of sufficient illumination intensity to be seen in low ambient lighting conditions, that is to say, little or essentially no light. Generally, such intensity is designed to not interfere with the user's sleep.

In some embodiments the illumination intensity is adjustable and can be turned off, while in still other embodiments such illumination is automatically controlled by an illumination controlling means (not shown) that has the capability of sensing ambient lighting for the purpose of providing appropriate illumination of activation means 20. In other embodiments in accordance with the present invention, activation means 20 is not illuminated, but rather it is formed having a texture that is readily identifiable by touch. In such embodiments, an optional illuminated portion or region of upper surface 12 can be provided for locating device 10 in darkened ambient conditions, for example an illuminated potion of upper surface 12 surrounding means 20. It will be understood that the various configurations of activation means 20 described above are directed toward providing for readily locating means 20 and device 10 in the low ambient lighting conditions likely to be found when a person wakes from a dream. Thus such configurations advantageously allow such a person to readily activate dream recollection recording device 10 and record a dream recollection by engaging activation means 20 without the problems associated with many of the previously known recording methods.

Dream recollection recording device 10 also includes an audio recording and playback means or apparatus (not shown), which encompasses at least a microphone, a speaker, a memory component, a device control component and an electrical power component. The microphone may be of any type for recording a person's voice. While generally located just below upper surface 12, the microphone can be located anywhere within housing 14. Typically, it is advantageous to locate the microphone proximate sound accepting region 24, depicted in FIG. 1 within the upper surface 12, so that it most efficiently picks up the user's voice. In some embodiments of the present invention, the microphone's sensitivity is adjustable to accommodate the different speaking levels of users, while in other embodiments such sensitivity is fixed to a level sufficient to successfully record a typical user's voice when speaking at a low volume.

The speaker of the playback means may be of any type for playing back or emanating sound. While generally located just below upper surface 12, it can be located anywhere within housing 14. Typically, it is advantageous to locate the speaker proximate the sound emitting region 22, depicted in FIG. 1 within the upper surface 12, so that the speaker most efficiently plays back the user's voice. In some embodiments of the present invention, the volume or playback level of the speaker is adjustable to accommodate different ambient sound levels during playback, while in other embodiments such playback level is fixed to a predetermined level.

The memory component generally encompasses one or more Random Access Memory (RAM) devices, but may alternately be any device or structure capable of storing the user's dream recollections. While it is generally advantageous for dream recollections to be stored in a digital format, it will be understood that embodiments in accordance with the present invention also include analog storage of such recollections. In addition, while generally the memory component or device 10 is fixed in capacity, some embodiments incorporate removable storage such as “Flash Memory” cards that are generally employed for the storage of digital images taken by digital cameras. Such removable storage media will not only provide for enlarging the storage capacity of device 10 so long detailed dream recollections can be recorded, but also provide for transferring recorded dream recollections to and from other devices, for example a computing means. In this manner, a large number of dream recollections can be cataloged for review and study.

The device control component of dream recollection recording device 10 serves to control the functions of the audio recording and playback means or apparatus. Thus when the user activates device 10 for recording by engaging activation means 20 a first time, the device control component provides for directing the appropriate recording and storage of the dream recollection. When the user deactivates device 10 by engaging activation means 20 a second time, the device control component provides for stopping the recording process and storing information that provides for playback of the recorded recollection. For example, where recollection storage is effected using digital memory, the control component positions the storage of the recollection at an available memory location and stores such location for playback. The device control component will interface to all functions of recording device 10 including but not limited to, the illumination of activating means 20, if any, recording and playback of recollections and any of the other functions of device 10 described herein. To effect such control, in some embodiments of the present invention, the control component will generally encompass a monolithic micro-controller integrated circuit, while in other embodiments such control device will encompass several discrete and/or integrated microelectronic devices coupled in an appropriate manner to result in the desired control functionality.

The electrical power component of device 10 is generally one or more replaceable batteries, although rechargeable batteries can also be employed. In addition, some embodiments of the present invention will encompass connectivity through the power component to an outside electrical source, for example to an appropriate transformer coupled to a common wall outlet.

Turning now to FIG. 2, the device of FIG. 1 is shown as seen from a side corresponding to a long axis of device 10. As depicted, a portion of upper surface 12 is shown encompassing portions of activation means 20 and sound emitting region 22, as well as sound accepting region 24. Also shown is a side portion 14 having playback controls 30, 32 and 34, volume control 36, port 38 for coupling dream recollection recording device to an external electrical energy source and erase control 40 for erasing one or more recorded dream recollections.

While, as previously described, activation means 20 serves to both activate and deactivate the recording function of device 10, the playback of dream recollections for the user's review and study is generally started and stopped using alternate control means. Playback controls 30, 32 and 34 are exemplary of such alternate control means. Thus, in an exemplary embodiment of the present invention controls 30, 32 and 34 are depressible buttons or switches coupled to the device control component of device 10. In such an embodiment, playback control 32 can be depressed a first time to start playback and a second time to stop playback, while playback controls 34 and 30 can be depressed to advance the playback forward and backward within a recollection record or to a subsequently or previously recorded record, respectively. It will be understood that playback control can be provided using controls 30, 32 and 34 configured in other manners or with other configurations of controls and that all such configurations are within the scope and spirit of the present invention. For example, one of controls 30, 32 and 34 can also serve to provide an erase function, that is a control that when activated will erase or delete one or several of stored dream recollections.

Still referring to FIG. 2, volume control 36, as depicted, can be either a rotary control or a slide control coupled to the device control component of device 10. That is to say, a control that when rotated or slid in a first direction increases the playback volume and when rotated or slid in a second, opposite direction decreases the volume. In some embodiments, such a rotary or slide control can also include a detent or stop that serves to turn power to the device off when the control is so positioned and on when the control is moved off of that position.

Port 38 is a receptacle for coupling to an external power source (not shown). As device 10 is generally powered using batteries, as previously described, such outside power source will typically couple to the source through a transformer or other means capable of reducing the voltage to device 10 to an appropriate level.

Erase control 40, as depicted, can be a depressible button or switch coupled to the device control component of device 10. In some embodiments of the present invention, such control 40 is engaged during the playback of a recollection to cause the erasure of that specific recollection. In this manner, erasure of multiple recollections can be accomplished by advancing through the recollections and engaging control 40 during the playback of each recollection for which erasure is desired. In some embodiments, an erasure safeguard feature can be provided where erase control 40 must be engaged multiple times or for an extended period of time to accomplish erasure of recollections.

While not described, embodiments in accordance with the present invention can have additional controls, features and/or status indicating devices incorporated within or coupled to the device control component. For example, some embodiments of device 10 have a port for coupling device 10 to a computing means for transferring recorded dream recollections thereto and thus clearing all or part of the memory means of device 10 for the recording of additional dream recollections. Exemplary coupling ports are those configured as USB (Universal Serial Bus) or Firewire ports although any other appropriately configured port can be employed. Embodiments of the present invention can also include an indicating means, such as a liquid crystal display or other low power display means, for indicating a variety of items. For example, such a display means can indicate the number of dream recollection records currently stored in the memory component or the number of records that can be subsequently stored in available memory or provide an alphanumeric record indicator. With such a display, when it is appropriate to erase or transfer out of the memory component currently stored dream recollections, the display can be used to select messages for erasure or transfer, for example by engaging. Other display means can advantageously indicate the current time of day, date, and or a set time for device 10 to automatically begin to record a user's dream recollection.

Embodiments of dream recollection recording device 10 can further includes a means for indicating when the record function has been activated. In some embodiments this indicating means can be a light, such as provided by a light emitting diode coupled to the control component and/or activation means 20, or it can be provided by activation means 20, itself. Thus where means 20 is normally illuminated to make locating device 10 easy in low ambient lighting conditions, such means can be configured to flash its illumination on and off to indicate that recording has begun.

When a person wishing to record dream recollections awakens from the sleeping state, embodiments in accordance with the present invention are readily located, for example by the illumination provided. When activation means 20 is engaged, the recording function is started. Where device 10 uses a digital memory storage means such as Flash memory, the control component is configured to conveniently locate storage of the recollection being recorded at an available memory location and to record such location in an appropriate manner. Upon completing the recording of the recollection, the person stops the recording function. This is generally accomplished by engaging activation means 20 a second time, although in some embodiments of the present invention, an alternate method of stopping the record mode is provided. Advantageously when the record mode is stopped, the control component locates the last memory location used and records such location in an appropriate manner so that a subsequent dream recollection can be recorded without interfering with any previously recorded recollections.

Thus, during the recording process, a file is essentially created for each dream recollection in the memory component of the dream recording device by any manner commonly known in the art of digital recording devices. Thus embodiments of the present invention can store a number of such recollection files that is limited only by the amount of recording time that may fit within the memory available in the memory component of device 10. Where an embodiment encompasses a display means for indicating the number of files recorded and/or the amount of memory remaining, the control component is configured to both determine such information and to provide it to the display.

Should the user wish to replay a recollection file, he or she would activate the replay mode of device 10, for example by engaging playback control 32. In embodiments in accordance with the present invention, generally playback of recollections begin with the first recorded recollection, however, where playback controls 30 and 34 are provided, the user can advance through recollections until the desired recollection is located. Where it is desired to adjust the volume of the playback, generally the user can perform such an adjustment by engaging volume control 36, if provided. It will be understood that users can repeat any one or several of the above interactions with the controls of dream recollection recording device 10 to playback one or more of the recorded recollections any number of times. For example in some embodiments, when the user reaches the last stored file, the most recently recorded file, he or she can engage control 32 a second time to hear playback of the first stored file, the oldest stored file.

Thus the stored recollection files can be replayed at the user's convenience to allow thoughtful examination and interpretation of the user's dream recollections. Using erase control 40 (FIG. 2), the user can selectively erase files, one at a time, by engaging the erase control during playback of the file to be erased, or cause all of the stored files to be erased by for example, engaging erase control 40 multiple times or for a predetermined amount of time. Alternately, and where a display means is provided that displays an alphanumeric file or record indicator, embodiments of the present invention can selectively erase files by advancing to the desired file, for example by engaging controls 30 and/or 34, and engaging erase control 40.

In some embodiments of the present invention a playback pause or stop control is provided. When such a control is engaged a first time during the playback of a recollection file, such playback is halted. Conveniently, such a control allows the user to reflect on the dream, or to discuss the dream with another person or to record thoughts about the dream in another medium, such as on paper or on a computing device. Generally, engaging the control a second time resumes playback. In some embodiments, such a pause or stop function can be engaged while in the recollection recording mode. Thus engaging the control during recording a first time halts such recording, and engaging the control a second time resumes the recording.

As mentioned above, some embodiments of the present invention include a means for transferring dream recollections to another device such as a computing device or computer. Such means will typically be either a USB or Firewire means. Where device 10 includes such transfer means, generally additional controls will be necessary to implement the transfer function. Similarly, where previously mentioned removable memory is employed, additional controls may also be needed to allow such memory to be removed from device 10 without damaging the recorded files. For example, generally such removable memory requires that power to such memory is turned off prior to its removal.

Embodiments in accordance with the present invention have been described hereinabove in language more or less specific as to structural and methodical features. It is to be understood, however, that the invention is not limited to the features shown and described above, since the means herein disclosed comprise only exemplary forms of putting the invention into effect. The invention is, therefore, claimed in any of its forms or modifications within the proper scope of the appended claims appropriately interpreted in accordance with the doctrine of equivalents.

1. A device for recording a dream recollection, comprising:
a housing unit comprising an upper surface;
an activation means having a portion thereof comprising a region of the upper surface, the portion being configured to be readily located in low ambient light conditions; and
an audio recording and playback means disposed within the housing unit and comprising a microphone, a memory component and a device control component, the audio recording and playback means coupled to the activation means so that a first engaging of the activation means causes activation of the audio recording and playback means to begin creation of a dream recollection file, and so that a second engaging of the activation means completes the creation of the dream recollection file by storing the file within the memory component and deactivating the audio recording and playback means.

2. The device of claim 1, where the portion of the activation means comprises more than half of the area of the upper surface.

3. The device of claim 1, where the portion of the activation means is illuminated.

4. The device of claim 1, where the portion of the activation means is textured.

5. The device of claim 1, where the memory component comprises digital memory.

6. The device of claim 1, where the memory component comprises removable Flash memory.

7. The device of claim 1, further comprising dream recollection playback controls.

8. The device of claim 7, where the dream recollection playback controls comprise a start/stop control, an advance forward control, an advance backward control and a volume control.

9. The device of claim 8, where the dream recollection playback controls further comprise a file erase control.

10 The device of claim 9, where the file erase control consists of a single button or switch.

11. The device of claim 1, further comprising dream recollection playback controls comprising a start/stop control, an advance forward control, an advance backward control, a volume control and a display means, the display means configured to provide an identifying indication of a dream recollection selected for playback.

12. The device of claim 11, further comprising a file erase control consisting of a single button or switch.

13. A method for recording dream recollections comprising:
providing an audio recording and playback device comprising an activation means, the activation means configured for readily locating the recording device in low ambient lighting conditions without interfering with a person's sleep and playback controls comprising a start/stop control, an advance forward control, an advance backward control, a volume control and an erase control;
upon waking from a sleep period, locating the audio recording device;
engaging the activation means causing the activation of a record function of the audio recording device for recording a dream recollection;
audibly recalling a dream; and
re-engaging the activation means causing the deactivation the record function of the audio recording device and storage of the dream recollection.

14. The method of claim 13, further comprising:
engaging the start/stop control causing the playback of a dream recollection to begin;
optionally engaging the advance forward control and/or the advance backward control to select a desired dream recollection for playback; and
re-engaging the start/stop control causing the playback of a dream recollection to stop.

15. The method of claim 13, further comprising after engaging the start/stop control to cause the playback of a dream recollection to begin engaging the erase control to delete the dream recollection being played back.

16. A device for recording and playing back dream recollections, comprising:
a housing unit comprising an upper surface;
an audio recording and playback means disposed within the housing unit and comprising a microphone, a speaker, a memory component and a device control component;
an activation means having a first portion thereof comprising a region of the upper surface, the first portion being configured to be readily located in low ambient light conditions, and a second portion thereof coupled to the audio recording and playback means, wherein the coupling of the second portion to the audio recording and playback means configured to turn on and turn off an audio recording function configured to store audio signals received through the microphone in the memory component;
dream recollection playback controls comprising a start/stop control, an advance forward control, an advance backward control and a volume control, the dream recollection playback controls individually coupled to the audio recording and playback means, wherein the individual couplings of the dream recollection playback controls are configured to start and stop playback of dream recollections stored in the memory component through the speaker, to advance forward and backward through recorded dream recollections to provide for selecting a desired dream recollection for playback and to adjust the volume of the playback.

17. The device of claim 16, where the memory component comprises removable or fixed Flash memory devices.

18. The device of claim 16, where the dream recollection playback controls further comprises a file erase control configured as a single button or switch.

19 The device of claim 16, further comprising a display means configured to provide an identifying indication for the dream recollection being recorded or being played back.

20. The device of claim 16, further comprising a USB or Firewire port for coupling to a computing means, the coupling configured for transferring data therebetween.
